(19) United States
(12) Patent Application Publication
(10) Pub. No.: US 2004/0131627 A1
(43) Pub. Date: Jul. 8, 2004

(54) Insect repellent

(76) Inventor: Chaim Werdyger, Lawrence, NY (US)

Correspondence Address: EDELL, SHAPIRO, FINNAN & LYTLE, LLC, 1901 RESEARCH BOULEVARD, SUITE 400, ROCKVILLE, MD, 20850, US

(21) Appl. No.: 10/474,517

(22) PCT Filed: Apr. 25, 2002
(86) PCT No.: PCT/US02/11851

(51) Int. Cl. A61K 39/00; A61K 39/38

(52) U.S. Cl. 424/184.1

(57) ABSTRACT

An insect repellent composition includes Hinoki oil and limonene. Other natural oils and substances can also be combined in the composition to enhance the efficacy of the composition for repelling insects. The composition can be incorporated into a spray, lotion or other suitable product and applied to a selected surface to repel insects from that surface. Alternatively, the composition can be incorporated into a polymer to form a variety of products having insect repelling characteristics.

### CROSS-REFERENCE TO RELATED APPLICATIONS

This application claims priority from U.S. Provisional Patent Application Serial No. 60/286,020, entitled “Insect Repellent” and filed Apr. 25, 2001. The disclosure of the above-mentioned provisional application is incorporated herein by reference in its entirety.

### BACKGROUND OF THE INVENTION

1. Technical Field

The present invention pertains to compositions-and corresponding methods of using the compositions for repelling insects. In particular, the present invention pertains to insect repellent compositions including naturally occurring substances.

2. Discussion of Related Art

Conventional insect repellents typically function by applying a repellent composition to a body part or other surface, where the repellent evaporates to create a shield extending about a few inches above the area of application. The presence of the repellent vapor confuses insects and affects their ability to locate the target host upon which the repellent is applied.

The specific composition and the amount and frequency of application of a repellent varies based upon the type of insect targeted as well as the risk of disease associated with that insect. For example, different mosquito repellent compositions are required to produce the desired level of repellency depending upon the type and location of mosquito encountered. In the U.S., mosquito repellents are typically formulated as outside comfort products to provide minor relief against mosquito bites for people engaging in outdoor activities (e.g., camping). However, in certain other countries, different forms of mosquitoes exist that are extremely aggressive and carry disease; A stronger and more effective repellent is required to repel these types of mosquitoes.

Many synthetic chemicals, such as DEET (N,N-diethyl toluamide), have been found to be effective in repelling mosquitoes and other insects. However, the problem with repellents utilizing these chemicals is that they can be readily absorbed through the skin when applied to the body of the user. Absorption of chemicals such as DEET can be dangerous, particularly for small children and wildlife, potentially leading to accidental poisonings and causing harm to the environment.

As a result of such dangers associated with utilizing synthetic chemicals in insect repellents, other non-toxic alternatives have been sought to obtain an effective repellent utilizing primarily essential oils or other natural ingredients. Many natural substances are known to repel insects, but these substances are typically effective for only a short period of time. In addition, the efficacy of many natural substances to repel certain insects may be lost during processing of the substances to form commercial products such as sprays, lotions or oils. Thus, previous attempts at combining two or more natural oils or other natural components to form an effective insect repellent having long lasting efficacy have typically met with failure.

### OBJECTS AND SUMMARY OF THE INVENTION

Accordingly, it is an object of the present invention to provide an insect repellent including naturally occurring substances or components that is effective in repelling insects for long periods of time.

It is another object of the present invention to provide an insect repellent including naturally occurring components that is effective in repelling aggressive and potentially disease carrying insects.

Yet another object of the present invention is to provide an insect repellent including naturally occurring components that is safe and non-toxic to humans and the environment.

The aforesaid objects may be achieved individually and/or in combination, and it is not intended that the present invention be construed as requiring two or more of the objects to be combined unless expressly required by the claims attached hereto.

According to the present invention, an insect repellent composition or formulation is provided including Hinoki oil and limonene. The combination of Hinoki oil and limonene at suitable amounts yields a composition that is highly effective in repelling mosquitoes and other insects for extended periods of time. For further effectiveness, other natural oils including, without limitation, peppermint, citronella, rosemary, clove, geranium, soy and mineral oils can be added to the composition.

The above and still further objects, features and advantages of the present invention will become apparent upon consideration of the following detailed description of specific embodiments thereof.

### DETAILED DESCRIPTION OF THE PREFERRED EMBODIMENTS

The Hinoki tree (Chamaecyparis obtusa) originates from Japan and has spread to parts of the west coast of the U.S. Many years ago, seeds from the Japanese Hinoki tree washed up into the Coquille Valley of southwestern Oregon and took root. The trees flourished over the years and spread southward approximately 100 miles into northern California, eastward approximately 80 miles, and northward approximately 30 miles. The American version of the Hinoki tree that has spread throughout parts of Oregon and California is commonly referred to as a Port Orford Cedar tree (Chamaecyparis lawsoniana). As used herein, the term “Hinoki oil” refers to tree oil extracted from the Japanese Hinoki tree, the American Hinoki or Port Orford Cedar tree, or combinations thereof. It has been found that Hinoki oil is effective in repelling insects of substantially all types without toxicity to humans and animals.

In producing the insect repellent composition of the present invention, Hinoki oil is extracted from the bark, leaves and wood of the tree utilizing any suitable method including, without limitation, cold expeller pressing, rolling, pressurization, solvent extraction and steam distillation. Cold expeller pressing is the preferred method of extraction of the Hinoki oil, as this extraction process ensures minimal alteration of the oil being collected.

The inventor has determined that the efficacy of extracted Hinoki oil as an insect repellent is considerably enhanced when combined with the citrus solvent limonene to form a uniform composition. The limonene further serves as a carrier for the Hinoki oil when the composition is integrated into any of the varying products described below. Preferably, the insect repellent includes Hinoki oil and D-limonene, an isomer of limonene which can be extracted from citrus peels and pulp utilizing a steam distillation or other suitable process. Limonene has been examined by the Environmental Protection Agency (EPA) and considered as presenting little or no risk relating to potential environmental and/or health impacts. Limonene also has certain insect repellent properties of its own. However, the inventor has determined that the combination of limonene with Hinoki oil provides a more sustained and greater level of insect repellency in comparison to either Hinoki oil or limonene used separately. In addition, the feature of the limonene serving as a carrier of the Hinoki oil further renders this combination particularly suitable as an insect repellent.

The composition including Hinoki oil and limonene can be utilized as an application product in the form of sprays, lotions, creams, salves or any other desired products for use on humans, animals and vegetation. Alternatively, the Hinoki oil combined with limonene can be incorporated into cosmetics and fragrances or impregnated into polymers for producing a variety of products as described below.

The insect repellent composition can be prepared utilizing any suitable method that combines the Hinoki oil and limonene to form a generally homogenous or uniform mixture. In an exemplary embodiment, a desired amount of each of the Hinoki oil and limonene are added into a vessel or container and mixed (e.g., via stirring or agitation) for a suitable period (e.g., a few minutes) to form the composition. The amount of each of the Hinoki oil and limonene may vary considerably while maintaining a desirable repellent efficacy of the composition. However, the inventor has determined that a composition of at least about 30% by weight of Hinoki oil and at least about 40% by weight of limonene yields a highly effective and long lasting insect repellent.

The composition can also include other natural oils to increase its efficacy as an insect repellent including, without limitation, peppermint oil, citronella oil, soybean oil, rosemary oil, clove oil and geranium oil. Each of these oils can be obtained from their respective plants utilizing any suitable extraction method including, without limitation, any of the extraction methods described above. In addition, vanillin can be added to the composition to control the rate of evaporation of the composition thus prolonging the duration of repellency of the composition after application. Any combination of these natural substances can be provided at selected amounts to enhance the ability of the composition to repel insects. Mineral oil can further be added to the composition, particularly when a number of additional natural substances are also added, to assist in obtaining a uniform mixture of the composition. A composition including Hinoki oil, limonene and any combination of additional natural or other substances is prepared in a substantially similar manner as described above, with the addition of components for combining in the composition being in any selected order.

An exemplary embodiment of the composition which is highly effective and long lasting in repelling insects includes the following components and their weight percentages (i.e., weight of component per weight of the composition): Hinoki oil (30%), D-limonene (40%), peppermint oil (6%), citronella oil (5%), soybean oil (5%), rosemary oil (3%), clove oil (2%), geranium oil (2%), vanillin (5%), and-mineral oil (2%).

The insect repellent compositions of the present invention can be formed as application products for covering a surface to be treated. Depending upon the type of product being prepared with the composition (e.g., lotion, cream, salve, etc.), suitable additives can be combined with the composition to achieve a desired consistency and viscosity. In addition, the application rate and amount of composition provided in the product can vary considerably based upon factors such as the type of application product and the application environment. The compositions can further be incorporated into cosmetics and perfumes or other fragrances. The compositions can be used in any suitable applicator including, without limitation, pump sprays, roll-on applicators, aerosols and misters. Alternatively, the compositions can be combined with paints, varnishes, waxes, and various other related products for applying to a surface such as the floor or wall.

In an exemplary embodiment of an application product, an insect repellent solution is prepared by combining one of the compositions of the present invention with an aqueous solution (e.g., distilled water) and a suitable emulsifier or surfactant (e.g., polyethylene glycol, stearates, laurates and like substances). Depending upon the application environment and type of insect targeted for repelling, the composition can be diluted to a desired concentration by combining a selected amount of the composition, water and emulsifier to form the solution. In particular, the inventor has determined that an insect repellent solution including one of the compositions of the present invention in an amount of at least about 5% by weight of the spray is highly effective in repelling insects.

Alternatively, the insect repellent compositions of the present invention can be incorporated into a polymer for manufacturing into a wide variety of final products. In an exemplary embodiment, one or more compositions are impregnated into a polymer in the molten state, resulting in a mixture of the polymer and compositions upon solidification of the polymer. Dispersion of the compositions within the solidified polymer can be controlled depending upon a particular application. For example, a composition can be dispersed relatively uniformly throughout the final polymer product or, alternatively, concentrated at one or more suitable locations and/or surfaces of the polymer product. Polymers suitable for incorporating the compositions include, without limitation, polyolefins (e.g., polyethylene, polypropylene, etc.), polyesters and polyamides.

The resultant polymers impregnated with the compositions of the present invention can be formed into films for applying to surfaces. Alternatively, the resultant polymers can be extruded and/or combined with other polymers and/or other suitable materials to form any desired final product. Examples of polymer products impregnated with the insect repellent composition include, without limitation, bracelets or bands (e.g., for the neck, wrist or ankle), collars for pets or animal livestock, flower pots or other gardening products, textile fiber materials (e.g., mats, rugs, coats, bedding materials, tents, etc.) and garbage bags.

In an exemplary embodiment, a polymer product incorporating one or more insect repellent compositions is formed into a wrist or ankle band. A suitable polymer for constructing the band is polyethylene. However, other polymers can also be utilized to construct such a band. The band is manufactured by impregnating the compositions into molten polyethylene and subsequently extruding the polyethylene to form the band. The band can be extruded as a closed toroidal or ring shaped member for securing around the wrist or ankle of a user. Alternatively, the band can be open and spiral shaped. Preferably, the insect repellent compositions are provided in an amount of about at least 15% by total weight of the band to render the wrist or ankle band highly effective in repelling various types of insects.

In another exemplary embodiment, a polyethylene garbage bag is impregnated with about at least 1% of one or more compositions by total weight of the garbage bag to render the garbage bag highly effective in repelling insects. A further embodiment includes an animal flea collar including a suitable support, such as a pouch, for a sheet of polyethylene or other suitable polymer impregnated with a selected amount of one or more of the compositions.

The compositions can further be hydrogenated utilizing any suitable method known in the art to a semi-solid waxy state for forming insect repelling candles, waxes, resins and the like.

The following examples are representative of the application and efficacy of some exemplary compositions of the present invention. One skilled in the art will recognize that these examples represent only some of the many ways of implementing the compositions of the present invention into various products repelling insects.

### EXAMPLE 1

An insect repellent composition was formed by combining the following components together to form a generally uniform mixture:

| Component | Weight Percentage |
|---|---|
| Hinoki oil | 30% |
| D-limonene | 40% |
| Peppermint oil | 6% |
| Citronella oil | 5% |
| Soybean oil | 5% |
| Rosemary oil | 3% |
| Clove oil | 2% |
| Geranium oil | 2% |
| Mineral oil | 2% |
| Vanillin | 5% |

A composition product was then prepared by combining the composition with an aqueous solution containing an emulsifier so that the composition formed about 10% by total weight of the product. The 10% composition product was tested utilizing laboratory-reared, blood-seeking, Aedes aegypti female mosquitoes having an age ranging from about 7-14 days.

Three subjects were tested in a laboratory environment by inserting their forearms into sleeve cages (23 cm×23 cm×57 cm), with each sleeve cage containing approximately 30 mosquitoes, where one arm of each subject was treated with approximately 1 ml of the 10% composition product per 600 cm² of the exposed forearm surface. Each subject initially inserted a non-treated forearm (hand protected with a latex glove) into a cage for approximately 30 seconds and then counted the number of mosquito bites occurring on the exposed forearm. Immediately after application of the 10% composition product to the treated forearm, the treated forearm (hand protected with a latex glove) of each subject was inserted into the same sleeve cage for approximately 30 seconds and then the number of mosquito bites were counted on the treated forearm. At 30 minutes, 60 minutes and 90 minutes after initial application, each subject again performed another 30 second exposure of the non-treated and treated forearms within the sleeve cage, and biting counts were recorded at each time interval. The subjects were further instructed not to wash their forearms during the 90 minute evaluation period.

A percent repellency was determined for the 10% composition product based upon the number of biting counts between non-treated forearms and treated forearms over the 90 minute evaluation period. Specifically, the percent repellency was determined utilizing the following formula: ((Number of mosquito bites for non-treated forearms)−(Number of mosquito bites for treated forearms))/(Number of mosquito bites for non-treated forearms)×100. The average number of mosquito bites occurring for non-treated forearms during a 30 second evaluation was 16.6 bites, clearly indicating a high level of biting activity. A mean percent repellency for each time interval based upon the results of each test subject is set forth in Table 1 below:

TABLE 1

| Time after application (minutes) | Percent Repellency |
|---|---|
| 0 | 100% |
| 30 | 95.5% |
| 60 | 50% |
| 90 | 40% |

The laboratory results indicate that the 10% composition product provides insect repellency protection for a sustained period of time after initial application.

### EXAMPLE 2

A composition product was prepared by combining the composition set forth in Example 1 with an aqueous solution containing an emulsifier so that the composition formed about 5%by total weight of the product. A field test of the 5% composition product was conducted in a study area consisting of a meadow bordering a large mixed deciduous and coniferous wood lot containing predominately maple, poplar, birch, tamarack, white cedar, and white pine trees. Adjacent the study area was a cattail marsh greater than 4 hectares in size and approximately 30 meters from the center of the study area. The cattail marsh provided a source of Aedes spp. and Anopheles spp. mosquitoes as well as Coquillettidia perturbans mosquitoes.

Three subjects were utilized for the field test. Each subject dressed in identical green overall, head nets and white cotton gloves with exposed forearms. Two of the subjects were not treated with any repellent and served as control subjects. To the forearms of the third subject was applied approximately 1 ml of the 5% composition product per 600 cm² of exposed forearm. The 5%composition product was applied immediately prior to the start of the biting count evaluation for the subjects.

The field test was conducted over a period of five evenings with biting count evaluations beginning at dusk (about 8:30 in the evening) to correspond with peak mosquito biting activity. Biting counts were not conducted on evenings when air temperature was below 12° C. , when winds were above about 25 kmph, or when rain occurred as these conditions limit mosquito host-seeking activity. Additionally, if biting activity fell below a minimum of one bite per 2 minutes per non-treated subject, the trial was postponed until the following evening. The mean air temperature during the five evening study was 19.4° C., with a wind speed generally below about 5 kmph.

The subjects were initially assigned to one of four positions on a grid located within the study area, with each grid position being at least 10 m from the other grid positions. Biting count evaluations were performed over a 30 minute period each evening, where each 30 minute period consisted of eight 3.5 minute time periods during which the frequency of mosquito bites were recorded for each individual. Between each 3.5 minute time period, a 15 second interval was provided for each subject to sequentially rotate to another grid position before starting another 3.5 minute time period for recording mosquito bites. Thus, biting count activity data was obtained for each subject at each grid position twice during each evening of the field test.

The subjects aspirated mosquitoes biting their forearms during each 3.5 minute time period. The mosquitoes were aspirated into 150 ml clear plastic vials, and, following the 3.5 minute time periods, the number of mosquitoes collected in each vial was counted.

Percent repellency was determined for the 5% composition product based upon the formula set forth in Example 1 (where the number of mosquitoes collected represented the number of mosquito bites for a treated or non-treated forearm). The mean number of mosquitoes biting treated and non-treated subjects over the five evening study was compared using analysis of variance and a Scheffe's comparison of means. The mean number of mosquitoes biting the forearms of the subjects during a thirty minute period and percent repellency are set forth in Table 2 below:

TABLE 2

| | Mean Number of | |
|---|---|---|
| | Mosquitoes Biting per | |
| Subject | a 30 Minute Period | Percent Repellency |
| Control/Non-treated | 33.10 ± 17.46 | — |
| Treated | 11.20 ± 5.59 | 66.2% |

The results of the tests conducted reveal that a composition product with as little as 5% of the composition by weight is highly effective in repelling mosquitoes. In addition, increasing the concentration of the composition in the product by as much as 10% by weight provides more than 95% protection against mosquitoes from targeting a surface in comparison to non-treated surfaces. The results further indicate that the compositions of the present invention are particularly effective in environments with aggressive mosquito biting activity.

From the foregoing description, it will be appreciated that the invention makes available a novel insect repellent composition containing natural components and that is highly effective in repelling insects for extended periods of time. It is believed that other modifications, variations and changes will be suggested to those skilled in the art in view of the teachings set forth herein. It is therefore to be understood that all such variations, modifications and changes are believed to fall within the scope of the present invention as defined by the appended claims.

What is claimed is:

1. A composition for use as an insect repellent comprising Hinoki oil and limonene.

2. The composition of claim 1, wherein the composition includes D-limonene.

3. The composition of claim 1, wherein the composition includes Hinoki oil in an amount of at least about 30% by weight of the composition.

4. The composition of claim 1, wherein the composition includes limonene in an amount of at least 40% by weight of the composition.

5. The composition of claim 1, further comprising at least one secondary oil selected from the group consisting of peppermint oil, citronella oil, soybean oil, rosemary oil, clove oil, geranium oil, mineral oil, and combinations thereof.

6. The composition of claim 1, further comprising vanillin.

7. The composition of claim 1, wherein the composition further comprises peppermint oil, citronella oil, soybean oil, rosemary oil, clove oil, geranium oil, mineral oil, and vanillin.

8. The composition of claim 7, wherein the composition includes an amount of Hinoki oil of about 30% by weight of the composition, an amount of limonene of about 40% by weight of the composition, an amount of peppermint oil of about 6% by weight of the composition, an amount of citronella oil of about 5% by weight of the composition, an amount of soybean oil of about 5% by weight of the composition, an amount of rosemary oil of about 3% by weight of the composition, an amount of clove oil of about 2% by weight of the composition, an amount of geranium oil of about 2% by weight of the composition, an amount of mineral oil of about 2% by weight of the composition, and an amount of vanillin of about 5% by weight of the composition.

9. An insect repellent product including the composition of claim 1, the product further including an aqueous solution and an emulsifier.

10. The product of claim 9, further including the composition in an amount of at least about 5% by weight of the product.

11. A product including a polymer impregnated with the composition of claim 1.

12. The product of claim 11, wherein the polymer is selected from the group consisting of polyolefins, polyesters, polyamides, and combinations thereof.

13. A band including a polymer impregnated with the composition of claim 1, wherein the band is configured to be secured about a wrist or an ankle.

14. A garbage bag including a polymer impregnated with the composition of claim 1.

15. A collar including a polymer impregnated with the composition of claim 1, wherein the collar is configured to be secured about a neck of an animal.

16. A method of making a composition for repelling insects, comprising:
combining a selected amount of Hinoki oil with a selected amount of limonene to form a substantially uniform blend of the Hinoki oil and the limonene.

17. The method of claim 16, wherein the selected amount of Hinoki oil is at least 30% by weight of the composition.

18. The method of claim 16, wherein the selected amount of limonene is at least about 40% by weight of the composition.

19. The method of claim 16, further comprising:
combining a selected amount of at least one secondary oil with the Hinoki oil and the limonene to form a substantially uniform mixture of the Hinoki oil, the limonene and the at least one secondary oil, wherein the at least one secondary oil is selected from the group consisting of peppermint oil, citronella oil, soybean oil, rosemary oil, clove oil, geranium oil, mineral oil, and combinations thereof.

20. The method of claim 16, further comprising:
impregnating the composition into a polymer.
